(19) United States
(12) Patent Application Publication
(10) Pub. No.: US 2004/0067561 A1
(43) Pub. Date: Apr. 8, 2004

(54) Process for the production of L-lysine using coryneform bacteria

(75) Inventors: Brigitte Bathe, Salzkotten (DE); Caroline Reynen, Melle (DE); Walter Pfefferle, Halle (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: Degussa AG, Duesseldorf (DE)

(21) Appl. No.: 10/629,551

(22) Filed: Jul. 30, 2003

Related U.S. Application Data
(60) Provisional application No. 60/401,752, filed on Aug. 8, 2002.

(30) Foreign Application Priority Data
Jul. 31, 2002 (DE) 102 35 029.9

(51) Int. Cl. C12P 13/08; C12N 1/21

(52) U.S. Cl. 435/115; 435/252.3

(57) ABSTRACT

A process for the production of L-lysine, comprising:

a) fermentation of the L-lysine producing coryneform bacteria that are at least sensitive to diaminopimelic acid analogues, in particular 4-hydroxydiaminopimelic acid;

b) enrichment of the L-lysine in the medium or in the bacterial cells; and optionally

c) isolation of the L-lysine or L-lysine-containing feedstuffs additive from the fermentation broth, so that ≧0 to 100% of the constituents from the fermentation broth and/or from the biomass are present,

and optionally bacteria are used in which in addition further genes of the biosynthesis pathway of L-lysine are enhanced, or bacteria are used in which the metabolic pathways that reduce the formation of L-lysine are at least partially switched off.

### CROSS-REFERENCE TO RELATED APPLICATIONS

This application claims priority to U.S. Provisional Application No. 60/401,752, filed Aug. 8, 2002 and DE (Germany)102 35 029.9, filed Jul. 31, 2002, which are both incorporated by reference.

### BACKGROUND OF THE INVENTION

1. Field of the Invention

The invention provides methods for producing amino acids by fermentation and bacterial strains for the production of amino acids by fermentation. The invention provides a process for the production of L-lysine using coryneform bacteria that are sensitive to at least one diaminopimelic acid analogue, in particular 4-hydroxy-diaminopimelic acid.

2. Description of the Related Art

L-amino acids, in particular L-lysine, are used in human medicine and in the pharmaceutical industry, in the foodstuffs industry and most particularly in animal nutrition.

It is known to produce amino acids by fermentation of strains of coryneform bacteria, in particular Corynebacterium glutamicum. On account of their great importance efforts are constantly being made to improve the production processes. Process improvements may relate to fermentation technology measures, such as for example stirring and provision of oxygen, or the composition of the nutrient media, such as for example the sugar concentration during the fermentation, or the working-up to the product form by for example ion exchange chromatography, or the intrinsic performance properties of the microorganism itself.

In order to improve the performance properties of these microorganisms methods involving mutagenesis, selection and choice of mutants are employed. In this way strains are obtained that are sensitive to antimetabolites such as for example the lysine analogue S-(2-aminoethyl)-cysteine, or that are auxotrophic for regulatorily important metabolites and that produce L-amino acids.

For some years recombinant DNA technology methods have also been employed to improve L-amino acid producing strains of Corynebacterium glutamicum, by amplifying individual amino acid biosynthesis genes and investigating the effect on L-amino acid production.

### BRIEF SUMMARY OF THE INVENTION

The inventors have been involved in devising new principles for improved processes for the fermentative production of L-lysine using coryneform bacteria.

### DETAILED DESCRIPTION OF THE INVENTION

Where L-lysine or lysine are mentioned hereinafter, this is understood to mean not only the bases, but also the salts such as for example lysine monohydrochloride or lysine sulfate.

The invention provides a process for the fermentative production of L-lysine using coryneform bacteria that are sensitive to diaminopimelic acid analogues, in particular 4-hydroxydiaminopimelic acid. The analogues are generally used in concentrations of ≧ (greater than/equal to) 3 to ≦ (less than/equal to) 30 g/l. This range includes all intermediate values and subranges, such as 3, 3.5, 4, 5, 6, 9, 10, 12.5, 15, 17.5, 20, 22, 25, 27.5, 29 and 30 g/l.

The invention also provides a process for the fermentative production of L-lysine using coryneform bacteria that already produce L-lysine and that are sensitive to diaminopimelic acid analogues, in particular 4-hydroxy-diaminopimelic acid.

This invention furthermore provides a process for the production of L-lysine in which the following steps are carried out:

a) fermentation by the L-lysine producing coryneform bacteria that are at least sensitive to diaminopimelic acid analogues, in particular 4-hydroxydiaminopimelic acid;

b) enrichment of the L-lysine in the medium or in the bacterial cells; and optionally

c) isolation of the L-lysine or L-lysine-containing feedstuffs additive from the fermentation broth, so that ≧0 to 100% of the constituents from the fermentation broth and/or from the biomass are present.

The invention similarly provides a process for the production of coryneform bacteria that are sensitive to diaminopimelic acid analogues, in particular 4-hydroxy-diaminopimelic acid.

The strains that are used produce L-lysine preferably already before the sensitivity to 4-hydroxydiaminopimelic acid.

The expression diaminopimelic acid analogues according to the present invention includes compounds such as

4-fluorodiaminopimelic acid,

4-hydroxydiaminopimelic acid,

4-oxodiaminopimelic acid, or

2,4,6-triaminopimelic acid.

The present invention also provides mutant coryneform bacteria producing L-lysine that are sensitive to one or more of the diaminopimelic acid analogues selected from the group comprising 4-fluorodiaminopimelic acid, 4-hydroxy-diaminopimelic acid, 4-oxodiaminopimelic acid or 2,4,6-triaminopimelic acid.

The invention moreover provides feedstuffs additives based on fermentation broth that contain L-lysine produced according to the invention and no or only traces of biomass and/or constituents from the fermentation broth formed during the fermentation of the L-lysine-producing microorganisms.

The term “traces” is understood to mean amounts of >0% to 5%.

The invention additionally provides feedstuffs additives based on fermentation broth, characterised in that

a) they contain L-lysine produced according to the invention, and

b) they contain the biomass and/or constituents from the fermentation broth in an amount of 90% to 100% that are formed during the fermentation of the L-lysine-producing microorganisms.

All of part of the fermented medium enriched with lysine may be used in feedstuffs, such as animal feed products. For instance, a liquid fraction or a solid fraction of the fermentation medium or broth, which is enriched in lysine, may be used to produce or enrich feedstuffs, including dairy, swine, beef, horse, poultry, aquaculture, insect, and pet foods.

Nutritional products, such as animal feeds, may be supplemented with L-lysine, or a solid or liquid fraction of the coryneform bacterium-fermented medium that comprises L-lysine. Methods for admixing or producing animal feeds, such as pelleted feeds, are known in the art. Such feeds may also contain other conventional feed ingredients containing proteins, carbohydrates or fats, oils, salts, vitamins, minerals, antioxidants, flavorings, fiber or bulking agents, binders, preservatives, antibiotics, hormones and growth promoting agents. Ingredients for animal feeds are known to those with skill in the art, however, specific feed ingredients are incorporated by reference to the Kirk-Othmer Encylcopedia of Chemical Technology, 4^{th} edition, see especially, vol. 10, pages 288-324.

L-lysine produced by fermentation with coryneform bacteria of the invention may also be used in nutritional products for humans, in pharmaceutical or cosmetic products, or for other industrial or commercial processes requiring L-lysine.

The process according to the invention serves for the fermentative production of L-lysine. The L-lysine (or fractions containing L-lysine) may optionally be isolated from a solid or liquid fraction of the fermented medium. For instance, L-lysine may be isolated from a solid or liquid fraction obtained by filtration of the fermented medium, by centrifugation of the fermented medium, or by precipitation of the fermented medium.

The microorganisms that are provided by the present invention can produce amino acids from glucose, sucrose, lactose, fructose, maltose, molasses, starch, cellulose or from glycerol and ethanol. These microorganisms may be representatives of coryneform bacteria, in particular of the genus Corynebacterium. Among the genus Corynebacterium there should in particular be mentioned the species Corynebacterium glutamicum, which is known to the specialists in this field for its ability to produce L-amino acids.

Suitable strains of the genus Corynebacterium, in particular of the species Corynebacterium glutamicum, are in particular the following known wild type strains

Corynebacterium glutamicum ATCC13032

Corynebacterium acetoglutamicum ATCC15806

Corynebacterium acetoacidophilum ATCC13870

Corynebacterium melassecola ATCC17965

Corynebacterium thermoaminogenes FERM BP-1539

Brevibacterium flavum ATCC14067

Brevibacterium lactofermentum ATCC13869 and

Brevibacterium divaricatum ATCC14020

and L-amino acid-producing mutants and/or strains produced therefrom,

such as for example the L-lysine-producing strains

Corynebacterium glutamicum FERM-P 1709

Brevibacterium flavum FERM-P 1708

Brevibacterium lactofermentum FERM-P 1712

Corynebacterium glutamicum FERM-P 6463

Corynebacterium glutamicum FERM-P 6464

Corynebacterium glutamicum ATCC 21513

Corynebacterium glutamicum ATCC 21544

Corynebacterium glutamicum ATCC 21543

Corynebacterium glutamicum DSM 4697 und

Corynebacterium glutamicum DSM 5715.

It has been found that coryneform bacteria that are sensitive to diaminopimelic acid analogues, in particular 4-hydroxydiaminopimelic acid, produce L-lysine in an improved manner.

In order to produce the coryneform bacteria according to the invention that are sensitive to 4-hydroxydiaminopimelic acid, mutagenesis methods described in the prior art are used.

For the mutagenesis there may be employed conventional in vivo mutagenesis processes using mutagenic substances such as for example N-methyl-N′-nitro-N-nitrosoguanidine or ultraviolet light (Miller, J. H.: A Short Course in Bacterial Genetics. A Laboratory Manual and Handbook for Escherichia coli and Related Bacteria, Cold Spring Harbor Laboratory Press, Cold Spring Harbor, 1992).

The coryneform bacteria that are sensitive to 4-hydroxy-diaminopimelic acid may be identified by plating out on nutrient media plates containing 4-hydroxydiaminopimelic acid. End concentrations of ca. 5 to 15 g/l, for example 10 g/l of 4-hydroxydiamino-pimelic acid in the nutrient medium are particularly suitable for this purpose. At this concentration mutants sensitive to 4-hydroxydiaminopimelic acid may be distinguished from the unchanged parent strains by a delayed growth. After selection the mutants sensitive to 4-hydroxydiaminopimelic acid exhibit an improved L-lysine production.

In addition it may be advantageous for the production of L-lysine, in addition to the sensitivity to 4-hydroxy-diaminopimelic acid to enhance, in particular overexpress, one or more enzymes of the respective biosynthesis pathway, glycolysis, anaplerosis, citric acid cycle, pentose phosphate cycle, amino acid export and optionally regulatory proteins. The use of endogenous genes is in general preferred.

The expressions “endogenous genes” or “endogenous nucleotide sequences” are understood to mean the genes or nucleotide sequences present in the population of a species.

The expressions “enhancement” and “to enhance” describe in this connection the increase of the intracellular activity of one or more enzymes or proteins in a microorganism that are coded by the corresponding DNA, by for example increasing the number of copies of the gene or genes, employing a strong promoter or a gene that codes for a corresponding enzyme or protein having a high activity, and optionally combining these measures.

By means of enhancement, in particular overexpression measures, the activity or concentration of the corresponding protein is generally raised by at least 10%, 25%, 50%, 75%, 100%, 150%, 200%, 300%, 400%, 500%, or even 1000% or 2000%, referred to the activity or concentration of the wild type protein and/or the activity or concentration of the protein in the starting microorganism.

Thus, for the production of L-lysine, in addition to the sensitivity to diaminopimelic acid analogues, in particular 4-hydroxydiaminopimelic acid one or more of the genes selected from the following group may be enhanced, in particular overexpressed:

the gene lysC coding for a feedback-resistant aspartate kinase (Accession No. P26512, EP-B-0387527; EP-A-0699759; WO 00/63388),

the gene dapA coding for dihydrodipicolinate synthase (EP-B 0 197 335),

the gene gap coding for glyceraldehyde-3-phosphate dehydrogenase (Eikmanns (1992). Journal of Bacteriology 174:6076-6086),

simultaneously the gene pyc coding for pyruvate carboxylase (DE-A-198 31 609, EP-A-1108790),

the gene zwf coding for glucose-6-phosphate dehydrogenase (JP-A-09224661, EP-A-1108790),

simultaneously the gene lysE coding for the lysine export protein (DE-A-195 48 222),

the gene zwal coding for the Zwal protein (DE: 19959328.0, DSM13115),

the gene lysA coding for diaminopimelic acid decarboxylase (Accession No. X07563),

the gene sigc coding for the sigma factor C (DE: 10043332.4, DSM14375),

the gene tpi coding for triose phosphate isomerase (Eikmanns (1992), Journal of Bacteriology 174:6076-6086) and

the gene pgk coding for 3-phosphoglycerate kinase (Eikmanns (1992), Journal of Bacteriology 174:6076-6086).

Furthermore it may be advantageous for the production of L-lysine, in addition to the sensitivity to 4-hydroxydiamino-pimelic acid, simultaneously to attenuate, in particular reduce the expression, of one or more of the genes selected from the following group:

the gene pck coding for phosphoenol pyruvate carboxykinase (DE 199 50 409.1, DSM13047),

the gene pgi coding for glucose-6-phosphate isomerase (U.S. Ser. No. 09/396,478, DSM12969),

the gene poxB coding for pyruvate oxidase (DE:1995 1975.7, DSM13114),

the gene deaD coding for DNA helicase (DE: 10047865.4, DSM14464),

the gene citE coding for citrate lysase (PCT/EP01/00797, DSM13981),

the gene menE coding for 0-succinylbenzoic acid CoA-ligase (DE: 10046624.9, DSM14080),

the gene mikE17 coding for the transcription regulator MikE17 (DE: 10047867.0, DSM14143) and

the gene zwa2 coding for the Zwa2 protein (DE: 19959327.2, DSM13113).

The term “attenuation” describes in this connection the reduction or switching off of the intracellular activity of one or more enzymes (proteins) in a microorganism that are coded by the corresponding DNA, by using for example a weak promoter or a gene or allele that codes for a corresponding enzyme with a low activity or inactivating the corresponding gene or enzyme (protein), and optionally combining these measures.

By means of these attenuation measures the activity or concentration of the corresponding protein is generally reduced to 0 to 75%, 0 to 50%, 0 to 25%, 0 to 10% or 0 to 5% of the activity or concentration of the wild type protein, and/or the activity or concentration of the protein in the initial microorganism. These ranges include all intermediate values and subranges, e.g. 0 to 10, 20, 30, 40, 50, 60, 70 or 75%.

Finally it may be advantageous for the production of L-lysine, in addition to the sensitivity to 4-hydroxy-diaminopimelic acid, also to switch off undesirable secondary reactions (Nakayama: “Breeding of Amino Acid Producing Microorganisms”, in: Overproduction of Microbial Products, Krumphanzl, Sikyta, Vanek (eds.), Academic Press, London, UK, 1982).

The microorganisms produced according to the invention are also covered by the invention and may be cultivated continuously or discontinuously in a batch process (batch cultivation) or in a fed-batch process (feed process) or repeated fed-batch process (repetitive feed process) for the purposes of producing L-lysine. A summary of known cultivation methods is described in the textbook by Chmiel (Bioprozesstechnik 1. Einführung in die Bioverfahrenstechnik (Gustav Fischer Verlag, Stuttgart, 1991)) or in the textbook by Storhas (Bioreaktoren und periphere Einrichtungen (Vieweg Verlag, Brunswick/Wiesbaden, 1994)).

The culture medium to be used must satisfy in a suitable manner the requirements of the respective strains. Descriptions of culture media for various microorganisms are contained in the handbook “Manual of Methods for General Bacteriology” of the American Society for Bacteriology (Washington D.C., USA, 1981).

As carbon source there may be used sugars and carbohydrates such as for example glucose, sucrose, lactose, fructose, maltose, molasses, starch and cellulose, oils and fats such as for example soy bean oil, sunflower oil, groundnut oil and coconut oil, fatty acids such as for example palmitic acid, stearic acid and linoleic acid, alcohols such as for example glycerol and ethanol, and organic acids such as for example acetic acid. These substances may be used individually or as a mixture.

As nitrogen source there may be used organic nitrogen-containing compounds such as peptones, yeast extract, meat extract, malt extract, corn steep liquor, soy bean flour and urea, or inorganic compounds such as ammonium sulfate, ammonium chloride, ammonium phosphate, ammonium carbonate and ammonium nitrate. The nitrogen sources may be used individually or as a mixture.

As phosphorus source there may be used phosphoric acid, potassium dihydrogen phosphate or dipotassium hydrogen phosphate or the corresponding sodium-containing salts. The culture medium must furthermore contain salts of metals, such as for example magnesium sulfate or iron sulfate, that are necessary for growth. Finally, essential growth promoters such as amino acids and vitamins may be used in addition to the aforementioned substances. Apart from these, suitable precursors may be added to the culture medium. The aforementioned starting substances may be added to the culture in the form of a single batch or may be fed in in an appropriate manner during the cultivation.

In order to regulate the pH of the culture basic compounds such as sodium hydroxide, potassium hydroxide, ammonia or ammonia water, or acidic compounds such as phosphoric acid or sulfuric acid are used as appropriate. In order to control foam formation antifoaming agents such as for example fatty acid polyglycol esters may be used. In order to maintain the stability of plasmids, suitable selectively acting substances, for example antibiotics, may be added to the medium. In order to maintain aerobic conditions, oxygen or oxygen-containing gas mixtures such as for example air are fed into the culture. The temperature of the culture is normally 20° C. to 45° C., and preferably 25° C. to 40° C. Cultivation is continued until a maximum amount of desired product has been formed. This target is normally achieved within 10 hours to 160 hours.

Methods for the determination of L-lysine are known from the prior art. The analysis may be carried out as described by Spackman et al. (Analytical Chemistry, 30, (1958), 1190) by anion exchange chromatography followed by ninhydrin derivatisation, or by reversed phase HPLC as described by Lindroth et al. (Analytical Chemistry (1979) 51: 1167-1174).

The concentration of L-lysine may optionally be adjusted to the desired value by the addition of L-lysine.

By means of the described processes it is possible to isolate coryneform bacteria that are sensitive to diaminopimelic acid analogues, in particular 4-hydroxy-diaminopimelic acid, and to produce L-lysine in an improved manner according to the described fermentation processes.

### EXAMPLE 1

### Screening for Mutants Sensitive to 4-hydroxydiaminopimelic Acid

The Corynebacterium glutamicum strain DSM13994 was produced by multiple, untargeted mutagenesis, selection and mutant selection from C. glutamicum ATCC13032. The strain DSM13994 is sensitive to the lysine analogue S-(2-aminoethyl)-L-cysteine and has a feedback-resistant aspartate kinase that is insensitive to inhibition by mixtures of lysine (or the lysine analogue S-(2-aminoethyl)-L-cysteine, 100 mM) and threonine (10 mM), whereas in contrast to this the activity of aspartate kinase in the wild type is inhibited up to 10% residual activity.

A pure culture of the strain was deposited as DSM 13994 on Jan. 16, 2001 at the German Collection for Microorganisms and Cell Cultures (DSM Brunswick) according to the Budapest Convention.

For screening on colonies that are sensitive to 4-hydroxy-diaminopimelic acid, the strain DSM13994 after UV mutagenesis (Sambrook et al., Molecular Cloning: A Laboratory Manual. 2^{nd} Edition, Cold Spring Harbor, New York, 1989) is plated out on LB agar plates containing 4-hydroxydiaminopimelic acid. The agar plates are supplemented with 10 g/l of 4-hydroxydiaminopimelic acid. The growth of the colonies is observed over 48 hours. At this concentration mutants sensitive to 4-hydroxydiamino-pimelic acid may be differentiated from the unaltered parent strain by a delayed growth. In this way a clone is identified that exhibits a substantially delayed growth compared to DSM13994. The strain is identified as DSM13994_Hdap_s.

### EXAMPLE 2

### Production of Lysine

The C. glutamicum strain DSM13994_Hdap_s obtained in Example 1 is cultured in a nutrient medium suitable for the production of lysine and the lysine content in the culture supernatant is determined.

For this purpose the strains are first of all incubated on agar plates for 24 hours at 33° C. Using this agar plate culture a preculture is inoculated (10 ml of medium in a 100 ml Erlenmeyer flask). The medium MM is used as medium for the preculture. The preculture is incubated for 24 hours at 33° C. at 240 rpm on a vibrator. Using this preculture a main culture is inoculated so that the initial optical density (OD −660 nm) of the main culture is 0.1 OD. The medium MM is also used for the main culture.

Medium MM

| | | |
|---|---|---|
| CSL | 5 | g/l |
| MOPS | 20 | g/l |
| Glucose (separately autoclaved) | 50 | g/l |
| Salts: | | |
| (NH₄) ₂SO₄ | 25 | g/l |
| KH₂PO₄ | 0.1 | g/l |
| MgSO₄ × 7 H₂O | 1.0 | g/l |
| CaCl₂ × 2 H₂O | 10 | mg/l |
| FeSO₄ × 7 H₂O | 10 | mg/l |
| MnSO₄ × H₂O | 5.0 | mg/l |
| Biotin (sterile filtered) | 0.3 | mg/l |
| Thiamine × HCl (sterile filtered) | 0.2 | mg/l |
| CaCO₃ | 25 | g/l |

CSL (Corn Steep Liquor), MOPS (morpholinopropanesulfonic acid) and the salt solution are adjusted with ammonia water to pH 7 and autoclaved. The sterile substrate and vitamin solutions as well as the dry autoclaved CaCO₃ are then added.

Culturing is carried out in a 10 ml volume in a 100 ml Erlenmeyer flask equipped with baffles. The culturing is carried out at 33° C. and 80% atmospheric humidity.

After 72 hours the OD is determined at a measurement wavelength of 660 nm with a Biomek 1000 instrument (Beckmann Instruments GmbH, Munich). The amount of lysine formed is determined by ion exchange chromatography and post-column derivatisation with ninhydrin detection, using an amino acid analyser from Eppendorf-BioTronik (Hamburg, Germany).

The result of the experiment is shown in Table 1

TABLE 1

| | OD | Lysine · HCl |
|---|---|---|
| Strain | (660 nm) | g/l |
| DSM13994 | 9.7 | 18.9 |
| DSM13994_Hdap_s | 7.5 | 19.6 |

Modifications and Other Embodiments

Various modifications and variations of the described bacterial strains, genes, compositions and methods as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the microbiological, fermenation, agricultural, chemical engineering, medical, biological, chemical or pharmacological arts or related fields are intended to be within the scope of the following claims.

Incorporation by Reference

Each document, patent application or patent publication cited by or referred to in this disclosure is incorporated by reference in its entirety. Any patent document to which this application claims priority is also incorporated by reference in its entirety. Specifically, U.S. Provisional Application No. 60/401,752, filed Aug. 8, 2002 and DE (Germany)102 35 029.9, filed Jul. 31, 2002, are hereby incorporated by reference.

1. A process for the production of L-lysine comprising:
a) fermenting a medium suitable for the production of L-lysine with an L-lysine producing coryneform bacterium that is sensitive to one or more diaminopimelic acid analogues for a time and under conditions suitable for the production of L-lysine in the medium or in the bacterium, and optionally,
b) isolating L-lysine from the fermentation medium or from the bacterium, so that ≧0 to 100% of the constituents from the fermentation broth and/or from the biomass are present.

2. The process of claim 1, wherein said bacterium has at least one gene of the biosynthesis pathway of L-lysine enhanced.

3. The process of claim 1, wherein said bacterium has one or more metabolic pathways that reduce the formation of L-lysine at least partially switched off.

4. The process of claim 1, wherein said coryneform bacterium has one or more of the following genes enhanced or overexpressed:
the gene lysC coding for a feedback-resistant aspartate kinase,
the gene dapA coding for dihydrodipicolinate synthase,
the gene gap coding for glyceraldehyde-3-phosphate dehydrogenase,
the gene pyc coding for pyruvate carboxylase,
the gene zwf coding for glucose-6-phosphate dehydrogenase,
simultaneously the gene lysE coding for the lysine export protein,
the gene zwal coding for the Zwal protein,
the gene lysA coding for diaminopimelic acid decarboxylase,
the gene sigc coding for the sigma factor C,
the gene tpi coding for triose phosphate isomerase, or
the gene pgk coding for 3-phosphoglycerate kinase.

5. The process of claim 1, wherein said bacterium has one or more genes from the following group attenuated:
the pck gene coding for phosphoenol pyruvate carboxykinase,
the pgi gene coding for glucose-6-phosphate-isomerase,
the gene deaD coding for DNA helicase,
the gene citE coding for citrate lysase,
the gene menE coding for O-succinylbenzoic acid CoA-ligase,
the gene mikE17 coding for the transcription regulator MikE17,
the gene poxB coding for pyruvate oxidase, or
the gene zwa2 coding for the Zwa2 protein.

6. The process of claim 1, wherein said coryneform bacterium is sensitive to 4-fluorodiaminopimelic acid.

7. The process of claim 1, wherein said coryneform bacterium is sensitive to 4-hydroxydiaminopimelic acid.

8. The process of claim 1, wherein said coryneform bacterium is sensitive to 4-oxodiaminopimelic acid.

9. The process of claim 1, wherein said coryneform bacterium is sensitive to 2,4,6-triaminopimelic acid.

10. The process of claim 1, wherein said bacterium is Corynebacterium glutamicum.

11. The process of claim 1, wherein in said bacterium is Corynebacterium glutamicum, which is sensitive to 4-hydroxydiaminopimelic acid.

12. The process of claim 1, wherein said bacterium is identified as Brevibacterium.

13. A mutant of a coryneform bacterium that produces L-lysine, which is sensitive to one or more of the diamino-pimelic acid analogues selected from the group consisting of 4-fluorodiamino-pimelic acid, 4-hydroxydiaminopimelic acid, 4-oxo-diaminopimelic acid and 2,4,6-triaminopimelic acid.

14. The process of claim 1, wherein said bacterium is a mutant of a coryneform bacterium that produces L-lysine, which is sensitive to one or more of the diaminopimelic acid analogues selected from the group consisting of 4-fluorodiaminopimelic acid, 4-hydroxydiaminopimelic acid, 4-oxo-diaminopimelic acid and 2,4,6-triaminopimelic acid.

15. A feedstuff additive produced by the process of claim 1, wherein said additive comprises the biomass and/or constituents from the fermentation broth formed during the fermentation of the L-lysine-producing microorganisms in an amount of not more than 0% to 5%.

16. A feedstuff additive produced by the process of claim 1, wherein said additive comprises the biomass and/or constituents from the fermentation broth formed during the fermentation of the L-lysine-producing microorganisms in an amount of 90% to 100%.

17. A liquid fraction of the fermented medium of claim 1.

18. A solid fraction of the fermented medium of claim 1.

19. A dairy, swine, beef, horse, poultry, aquaculture or pet feed comprising all or part of the fermentation medium produced by the process of claim 1.

20. A method for feeding an animal comprising administering a composition comprising the fermentation medium produced by the process of claim 1.

21. A method for making a feed comprising admixing L-lysine or a solid or liquid fraction comprising L-lysine, which is produced by the process of claim 1, with one or more other feedstuff ingredients.
